(19) United States
(12) Patent Application Publication
(10) Pub. No.: US 2005/0043702 A1
(43) Pub. Date: Feb. 24, 2005

(54) Disposable feminine panty and sanitary napkin

(76) Inventors: Kathy S. Houchen, Waldorf, MD (US); Rhonda A. Smith, Washington, DC (US)

Correspondence Address: Kathy S. Houchen;Rhonda A. Smith, 2017 37th Street, SE, #202, Washington, DC, 20020, US

(21) Appl. No.: 10/645,549

(22) Filed: Aug. 22, 2003

(51) Int. Cl. A61F 13/15

(52) U.S. Cl. 604/396

(57) ABSTRACT

A disposable undergarment comprises a front portion made of disposable material, a rear portion made of disposable material, and a crotch portion made of disposable material. The crotch portion includes a sanitary pad fixed to the undergarment in a pocket or pouch as part of the undergarment.

### FIELD OF INVENTION

This invention relates to a disposable, sexy looking, and comfortable undergarment that has a sanitary pad fixed (for example, sewn) into the undergarment to give a woman the confidence and assurance needed during her menstrual cycle.

### BACKGROUND OF INVENTION

Most undergarments that woman wear during the menstrual cycle are often unattractive undergarments that are used specifically for this period of the month. The undergarments are stained and never get clean in the washing machine, so often we end up after a few uses throwing those unattractive, stained undergarments in the trash. Even so, sanitary pads stuck to undergarments with an adhesive strip often do not provide the comfort and security to keep the sanitary pad attached to the undergarment that all women need during this sensitive time. Sanitary pads used with regular undergarments often leak through and move around in the undergarment. When women go to sleep with the regular sanitary pad and undergarment, the sheets usually get stained, or when sitting in a chair, the chair will often get stained. Skirts, dresses and pants get stained because of the insecurity of the regular sanitary pad and undergarment.

### SUMMARY OF INVENTION

This invention is a new wave in the way of how women will care for themselves during their menstrual cycle. The security and confidence of wearing a sexy, comfortable and disposable undergarment during a woman's menstrual cycle is a lot of wishes come true. This disposable feminine undergarment comes in fabrics such as, for example, satin, silk and cotton.

A worry free undergarment during a woman's menstrual cycle is virtually unheard of. This invention comes in brief or bikini styles of all patterns and colors.

Whether you are flowing heavy or light, you can still look sexy and feel assured that the sanitary pad fixed into the crotch section (for example, by sewing or fusing) is absorbent and will keep you protected from leakage and embarrassment. The crotch portion of this invention is in three parts, 1) the top absorbent lining, 2) the absorbent sanitary pad and 3) the leak-proof bottom lining. The top absorbent lining and the leak-proof bottom lining are fixed (for example by sewing or fusing) around the sanitary pad to make sure that the sanitary pad does not move around when the wearer moves around. The sanitary pad fits neatly and securely in the enclosed top and bottom linings. The top of the undergarment itself is an elastic waist band integrally knit at the upper portion of the undergarment for a snug and secure feeling around the wearer's waist or bikini level area.

The disposable sexy looking undergarment can easily be tossed in the trash after use. No more trying to get stains out of panties, sheets or furniture.

### DESCRIPTION OF THE DRAWINGS

The present invention may be better understood from the following detailed description and drawings of some preferred embodiments, in which:

FIG. 1 is the front view of a brief style undergarment including the sewn/fused in crotch of construction of the present invention;

FIG. 2 is the rear view of a brief style undergarment including the sewn/fused in crotch of construction of the present invention;

FIG. 3 is the front view of a bikini style undergarment including the sewn/fused in crotch of construction of the present invention;

FIG. 4 is the rear view of a bikini style undergarment including the sewn/fused in crotch of construction of the present invention;

FIG. 5 shows the absorbent sanitary pad that is elongated through the crotch of the undergarment;

FIG. 6 shows the top absorbent cover of the crotch lining which is sewn/fused over the top of the sanitary napkin;

FIG. 7 shows a thin, for example, non-thermoplastic leak-proof layer to be placed beneath the pad for enclosing the sanitary pad; and

FIG. 8 shows an elastic waist band integrally knit with the upper end of the undergarment.

### DETAILED DESCRIPTION OF PREFERRED EMBODIMENTS

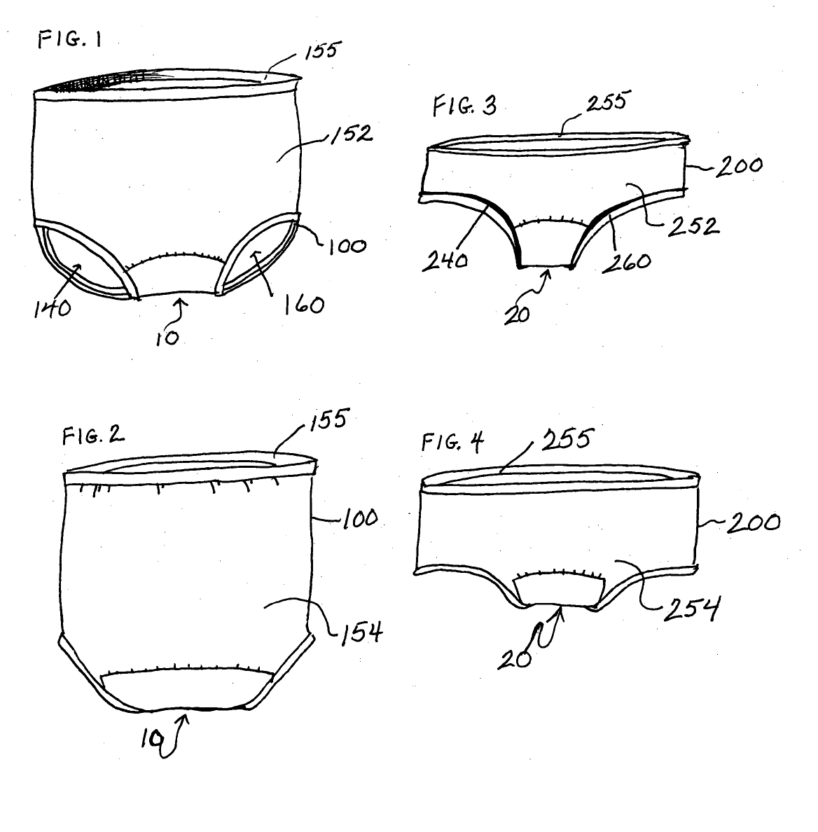

FIGS. 1 to 6 illustrate preferred embodiments of the invention. FIG. 1 is a front view of a brief-style undergarment 100 and FIG. 2 is a rear view of the brief-style undergarment 100.

As shown in FIGS. 1 and 2, undergarment 100 includes a front portion 152, a rear portion 154, a waist portion 155, leg openings 140 and 160, and a crotch portion 10. The leg openings may or may not include elastic material. The front portion and the rear portion do not include absorbent padding. Portions 152, 154 and 155 can be made from different pieces of material or from the same piece of material.

FIG. 3 is a front view of a bikini-style undergarment 200 and FIG. 4 is a rear view of the bikini-style undergarment 200. As shown in FIGS. 3 and 4, undergarment 200 includes a front portion 252, a rear portion 254, a waist portion 255, leg openings 240 and 260 and a crotch portion 201. Portions 154, 252, 254, and 255 can be made from different pieces of material or from the same piece of material.

In one embodiment of the invention, portions 152, 252 and 254 are made from soft, breathable polypropylene (hospital gown material), such as in disposable boxers and briefs as offered by Magellan (Fall 2003 Reference Edition, page 17, items MU230, MU235, and MU240). The entire contents of this catalog relating to undergarment materials is incorporated herein by reference.

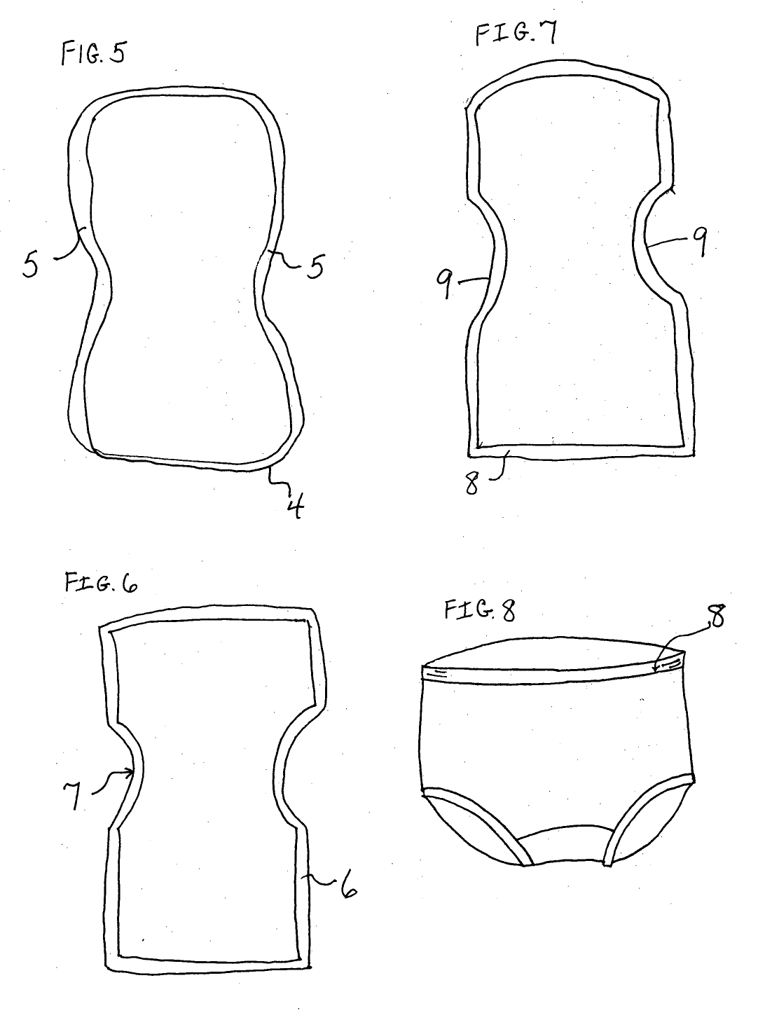

Crotch sections 10 and 20 of undergarments 100 and 200 have a similar design. This design will be described in connection with FIGS. 5 to 7. In the preferred embodiments, the crotch section comprises three parts: (1) an absorbent pad or sanitary napkin 4, shown in FIG. 5; (2) absorbent top lining portion 6, shown in FIG. 6; and (3) a leak-proof bottom portion or lining 8, shown in FIG. 7. Each of these portions 4, 6, and 8 have leg cutouts 5, 7 and 9, respectively. The pad 4 is thus sandwiched in a pocket or pouch sealed by top portion lining 6 and bottom lining 8. The pad is part of the garment itself and thus can not shift significantly or come loose. The pad is fixed to the undergarment. “Fixed” means that it is attached to the garment, for example by sewing or fusing such that it will not move in everyday use. Shown in FIG. 8 is the top portion of the undergarment integrally knit with an elastic band such as described in U.S. Pat. No. 4,150,554. The entire content, of this patent are incorporated herein by reference.

Undergarments 100 and 200 are fabricated from disposable materials, made of cotton, polyester or other natural or synthetic materials. In some cases, inexpensive grades of silk or satin may be used. In this context, the term “disposable” means an inexpensive grade of material which is intended to be used once and then thrown away or recycled. In these preferred embodiments, the waist portion also includes elastic material, for example, U.S. Pat. No. 4,150,554. Suitable materials are described in, for example, U.S. Pat. No. 6,172,276 (absorbent material for sanitary napkins) and U.S. Pat. No. 4,892,534 (bodyside liners). The bottom portion of the undergarment is a leak-proof sheet, with at least one absorbent layer, the leak-proof sheet has a porous aggregate of fibers integrated therewith, for example, U.S. Pat. No. 5,069,677). The entire contents of these documents are incorporated herein by reference.

In the preferred embodiments, the various portions are joined together by sewing. Stitching around the perimeter of the top and bottom portions that are used to affix the top and bottom portions to the rest of the garment also serves to keep the pad in place and other techniques such as heat fusing or gluing with a strong adhesive may be used to join the portions. Also, as mentioned above, the various portions (such as the front and rear portions) may be formed as one piece of material, in which event joining is unnecessary. An elastic waist band is knitted to the top portion of the undergarment. The terms “front portion: and “rear portion” refer to the front part of the garment and the rear part of the garment, even if the front part and the rear part are formed of one piece of material. The “top portion” refers to the opening at the top of the garment.

The top portion of the sanitary pad and the bottom portion may also be formed as one piece of material fixed to the remainder of the garment.

The invention is, of course, not limited to the preferred embodiments, described above. Variations and modifications will occur to others, in light of the above teachings. Thus, the invention is defined with reference to the claims set forth below.

This invention thus provides a sexy, comfortable, and disposable protective undergarment which has a sanitary napkin fixed (for example by sewing) in the crotch. This sexy undergarment may come in brief or bikini styles, in various colors and can be made of various fabrics, such as satin, silk or cotton. The crotch is elongated and has the sanitary pad inside. An absorbent layer covers the top of the sanitary pad in the crotch to keep wetness away from the body, while a thin leak-proof moisture absorbent layer covers the bottom of the pad to give the female even more security from leakage. The sanitary pad is fixed into the crotch of the undergarment to keep the pad from moving around as you sleep, walk or just enjoy other activities. This comfortable undergarment provides ultimate feminine security during a women's menstrual cycle, whether the female menstrual cycle flows heavy or light. This disposable undergarment provides a sanitary napkin portion. The sanitary napkin portion may be ultra thin, or medium or super absorbent. Embarrassing leakage in this sexy undergarment will not occur, nor will the wearer worry about the sanitary pad moving about; and most of all this undergarment is disposable, so no more worrying about washing soiled panties again. This undergarment provides confidence and style to females during menstrual cycles.

1. An undergarment comprising:
a front portion made of disposable material; a rear portion, connected to the front portion and made of disposable material;
an elastic waist band is integrally knit with the top portion of the undergarment,
wherein the crotch portion connected to the front portion and the rear portion and made of disposable material;
a crotch portion includes a sanitary pad fixed to the undergarment as part of the undergarment; and
the front portion and the rear portion do not include padding.

2. An undergarment as set forth in claim 1, wherein the front portion and the rear portion define a brief-style undergarment.

3. An undergarment as set forth in claim 1, wherein the front portion and the rear portion define a bikini-style undergarment.

4. An undergarment as set forth in claim 1, wherein the crotch portion further comprises a top portion and a bottom portion.

5. An undergarment as set forth in claim 4, wherein the sanitary pad is sandwiched between the top portion and the bottom portion.

6. An undergarment as set forth in claim 1, wherein the sanitary pad is fixed to the undergarment by sewing/fusing.

7. An undergarment as set forth in claim 1, wherein the sanitary pad is fixed to the undergarment by fusing.

8. An undergarment as set forth in claim 6, wherein stitches at least partly surround the sanitary pad to fix the sanitary pad to the undergarment.

9. An undergarment as set forth in claim 7, wherein fusing at least partly surrounds the sanitary pad to fix the sanitary pad to the undergarment.

10. An undergarment as set forth in claim 1, wherein the top portion is an elastic waist band integrally knit with the upper portion of the undergarment.
